(19) United States
(12) Patent Application Publication
(10) Pub. No.: US 2015/0238447 A1
(43) Pub. Date: Aug. 27, 2015

(54) BETA-HYDROXY-BETA-METHYLBUTRYIC ACID-CONTAINING COMPOSITIONS AND USES THEREOF

(71) Applicant: ABBOTT LABORATORIES, Abbott Park, IL (US)

(72) Inventors: Shreeram Sathyavageeswaran, Singapore (SG); Rema Vazhappilly, Singapore (SG); Tapas Das, Worthington, OH (US); Richard Gelling, Singapore (SG)

(73) Assignee: ABBOTT LABORATORIES, Abbott Park, IL (US)

(21) Appl. No.: 14/428,535

(22) PCT Filed: Sep. 17, 2013
(86) PCT No.: PCT/US2013/060120

§ 371 Date: Mar. 16, 2015

Related U.S. Application Data
(60) Provisional application No. 61/701,794, filed on Sep. 17, 2012.

(51) Int. Cl. A61K 31/19; A23L 2/52; A61K 47/36; A23L 1/305; A61K 47/42; A61K 47/44

(52) U.S. Cl. CPC A61K 31/19; A61K 47/42; A61K 47/44; A61K 47/36; A23L 1/3051; A23L 2/52

(57) ABSTRACT

Disclosed are compositions, such as liquid and solid nutritional compositions, that contain beta-hydroxy-beta-methylbutyric acid and optionally at least one protein, carbohydrate, fat, or a combination of protein, carbohydrate, and fat. Also disclosed are methods of using the compositions for improving physical endurance, locomotion, mobility and cognition in an individual, including a pediatric individual.

### CROSS-REFERENCE TO RELATED APPLICATION

This application claims priority to and any other benefit of U.S. Provisional Application No. 61/701,794 filed Sep. 17, 2012 and entitled BETA-HYDROXY-BETA-METHYLBUTRYIC ACID-CONTAINING COMPOSITIONS AND USES THEREOF FOR IMPROVING ENDURANCE,” the entire disclosure of which is incorporated by reference herein.

### TECHNICAL FIELD

The present disclosure relates to compositions including beta-hydroxy-beta-methylbutyric acid and methods for improving physical endurance, physical activity, and/or academic performance using nutritional compositions. More specifically, the present disclosure relates to compositions comprising beta-hydroxy-beta-methylbutyric acid (HMB) for use in increasing dopamine homeostasis in the brain, improving locomotion, mobility, cognition, and endurance.

### BACKGROUND OF THE DISCLOSURE

Physical endurance in children is of great importance to overall health and well being. A high fat diet has been demonstrated to impair serine/threonine-specific protein kinase (Akt) signaling by reducing phosphorylation of Akt which in turn has been demonstrated to disrupt dopamine (DA) homeostasis in brain resulting in impaired locomotor or exploratory activity. See, R. L. Barry, High-Fat Diet Modulates Dopaminergic Network Activity: An Analysis of Functional Connectivity, Proc. Intl. Soc. Mag. Reson. Med. 18 (2010). Efforts to improve physical endurance by increased muscle mass using leucine as a dietary supplement have become popular. The use of leucine, however, is not particularly well suited for use in dietary supplements that the user can taste. In particular, leucine imparts a bitter taste that is not compatible with a favorable flavor profile in liquid or solid compositions. Furthermore, leucine is difficult to disperse in liquid compositions, thereby complicating the manufacture of leucine-containing compositions. Finally, to achieve improvement similar to that shown herein, higher levels of leucine would be required, thereby compounding the problem of bitterness and manufacture of liquid compositions containing leucine at effective concentrations.

As such, there is a need for nutritional compositions without high leucine levels and methods of using such nutritional compositions for improving physical endurance, physical activity, and in some instances, academic performance in pediatric individuals.

### SUMMARY

It has been unexpectedly found that the use of a composition including beta-hydroxy-beta-methylbutyric acid (HMB) significantly improves physical endurance in a pediatric population. It has also been found that such improvement can be obtained without requiring higher concentrations of leucine in the composition. Additionally, it has been found that that such improvement can be obtained when the composition is substantially free of free leucine.

Furthermore, it has been discovered that HMB enhances sensorimotor skills and/or cognitive functioning in obese children or adults through the modulation of Akt phosphorylation resulting in the restoration of dopamine (DA) homeostasis in brain. It has also been found that HMB increases dopamine homeostasis in the brain of obese individuals, improving locomotion, mobility and cognition in the individual.

Accordingly, the HMB-containing compositions and methods of using same, as disclosed herein in accordance with the general inventive concepts, contribute to improved endurance in pediatric individuals. Accordingly, the present disclosure describes nutritional compositions containing HMB and methods of using the compositions to improve endurance in pediatric individuals. Further, the present disclosure describes nutritional compositions comprising HMB for use in increasing dopamine homeostasis in the brain of an obese individual resulting in improved or enhanced locomotion, mobility, or cognition in the individual.

A first embodiment exemplary is directed to a pediatric nutritional composition comprising at least one of protein, carbohydrate, fat, or a combination thereof and from 0.1% to 20% beta-hydroxy-beta-methylbutyric acid by weight. This nutritional composition is capable of improving physical endurance in a pediatric individual.

In a second exemplary embodiment, a nutritional composition comprising beta-hydroxy-beta-methylbutyric acid for use in increasing dopamine homeostasis in the brain of an obese individual is provided. In this embodiment, use of the nutritional composition results in improving at least one of locomotion, mobility, and cognition in the obese individual.

In a third exemplary embodiment, a composition comprising an effective amount of beta-hydroxy-beta-methylbutyric acid for use in improving physical endurance in a human subject between 1 year of age and 13 years of age is provided. The composition may be a nutritional composition. In this embodiment, the use results in improving physical endurance in the subject.

In a fourth exemplary embodiment, a pediatric nutritional composition comprising calcium beta-hydroxy-beta-methylbutyric acid, whey protein, casein protein, soy protein, medium chain triglyceride oil, and fructooligosaccharides is provided.

### BRIEF DESCRIPTION OF THE DRAWINGS

FIG. 1 is a graph depicting the average distance traveled on the treadmill by mice, according to Example 1.

FIGS. 2a-2d are graphs depicting exploratory activity in mice, according to Example 2.

### DETAILED DESCRIPTION

Compositions, along with their methods of use, which include beta-hydroxy-beta-methylbutyric acid (HMB) for improving endurance in the pediatric population and improving locomotion, mobility and cognition in obese individuals are disclosed herein. The nutritional compositions and related methods as described herein provide pediatric individuals with a method of improving endurance. The compositions disclosed herein contain beta-hydroxy-beta-methylbutyric acid for use in increasing dopamine homeostasis in the brain of an obese individual resulting in improved locomotion, mobility and cognition in the individual.

A first exemplary embodiment is directed to a pediatric nutritional composition comprising at least one of protein, carbohydrate, fat, and combinations thereof and from 0.1% to 20% beta-hydroxy-beta-methylbutyric acid by weight. The nutritional composition of the first embodiment is capable of improving physical endurance in a pediatric individual.

In a exemplary second embodiment, a nutritional composition comprising beta-hydroxy-beta-methylbutyric acid for use in increasing dopamine homeostasis in the brain of an obese individual is provided. In this embodiment, the use of HMB results in the improvement of at least one of locomotion, mobility and cognition in the individual.

In a third exemplary embodiment, a composition comprising an effective amount of beta-hydroxy-beta-methylbutyric acid for use in improving physical endurance in a human subject between 1 year of age and 13 years of age is provided. In the third embodiment, the use results in improvement of physical endurance in the subject.

In a fourth exemplary embodiment, a pediatric nutritional composition comprising calcium beta-hydroxy-beta-methylbutyric acid, whey protein, casein protein, soy protein, medium chain triglyceride oil, and fructooligosaccharides is provided.

The term “endurance” as used herein means the time span between the beginning of physical activity by an individual and the termination of such activity because of exhaustion. The term “improve endurance” and variations thereof as used herein, unless otherwise specified, means a reduction of time between the beginning of physical activity by an individual and the termination of such activity because of exhaustion, or, in other words, increasing time to exhaustion or muscle failure.

The term “locomotion” as used herein means the act of moving from place to place.

The term “mobility” as used herein means the ability to move a body or body part from place to place.

The term “cognition” as used herein unless otherwise specified, means the mental processes involved in gaining knowledge and comprehension, including thinking, knowing, remembering, judging, and problem solving, including higher-level functions of the brain which encompass language, imagination, perception, and planning.

The term “high fat diet” as used herein means a diet where an individual receives at least 45% of his total caloric intake from fat.

The terms “obese” and “obese individual” as used herein mean a body mass index of 30 or higher for an adult or a body mass index-for-age of equal to or greater than the 95th percentile for pediatric individuals and teens.

The terms “free” and “substantially free” mean the selected composition or method contains or is directed to less than a functional amount of the ingredient or feature, typically less than 0.1% by weight, and also including zero percent by weight, of such ingredient or feature. The nutritional compositions and methods herein may also be “free of” or “substantially free of” any optional or other ingredient or feature described herein provided that the remaining composition still contains the requisite ingredients or features as described herein.

The terms “fat,” “oil,” and “lipid” as used herein, unless otherwise specified, are used interchangeably to refer to lipid materials derived or processed from plants or animals. These terms also include synthetic lipid materials so long as such synthetic materials are suitable for oral administration to humans.

The terms “nutritional formula,” “nutritional product,” and “nutritional composition,” are used interchangeably herein and, unless otherwise specified, refer to nutritional liquids, nutritional semi-liquids, nutritional solids, nutritional semi-solids, nutritional powders, nutritional supplements, and any other nutritional food product form as known in the art. The nutritional powders may be reconstituted to form nutritional liquids, which comprise one or more of protein, carbohydrate, fat, and are suitable for oral consumption by a human.

The term “nutritional liquid” as used herein, unless otherwise specified, refers to nutritional products in ready-to-drink liquid form, concentrated form, and nutritional liquids made by reconstituting the nutritional powders described herein prior to use.

The term “nutritional powder” as used herein, unless otherwise specified, refers to nutritional products in flowable or scoopable form that can be reconstituted with water or another aqueous liquid prior to consumption and includes both spray dried and drymixed/dryblended powders.

The term “nutritional solid” as used herein, unless otherwise specified, refers to products that are generally solid in nature such as cereals, bars, baked goods, and the like.

The terms “pediatric” and “pediatric individual” are used interchangeably herein to refer to individuals from the age of greater than 1 year to 13 years or less, including the age of greater than 1 year to 10 years or less.

The terms “teen” and “teenager” are used interchangeably herein to refer to individuals from the age of 13 years to 19 years.

The term “pediatric nutritional composition” as used herein refers to nutritional products that are designed specifically for consumption by a pediatric individual.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

Any reference to a singular characteristic or limitation disclosed herein shall include the corresponding plural characteristic or limitation, and vice versa, unless otherwise specified or clearly implied to the contrary by the context in which the reference is made.

Any combination of method or process steps as used herein may be performed in any order, unless otherwise specifically or clearly implied to the contrary by the context in which the referenced combination is made.

The nutritional compositions and methods may comprise, consist of, or consist essentially of the elements and features of the disclosure described herein, as well as any additional or optional ingredients, components, or features described herein or otherwise useful in a nutritional application.

### Beta-Hydroxy-Beta Methylbutyric Acid (HMB)

The composition of the first exemplary embodiment is a pediatric nutritional composition. The composition comprises at least one of a protein, carbohydrate, fat, and combinations thereof, and from about 0.1% to about 20% beta-hydroxy-beta-methylbutyric acid by weight. The nutritional composition is capable of improving physical endurance in a pediatric individual. The composition may comprise 80% to 99.9% fat in addition to about 0.1% to about 20% beta-hydroxy-beta-methylbutyric acid by weight. The composition may comprise 80% to 99.9% protein in addition to about 0.1% to about 20% beta-hydroxy-beta-methylbutyric acid by weight. The composition may comprise 80% to 99.9% carbohydrate in addition to about 0.1% to about 20% beta-hydroxy-beta-methylbutyric acid by weight. The composition may comprise a combined total of 80% to 99.9% fat plus protein in addition to about 0.1% to about 20% beta-hydroxy-beta-methylbutyric acid by weight. The composition may comprise a combined total of 80% to 99.9% fat plus carbohydrate in addition to about 0.1% to about 20% beta-hydroxy-beta-methylbutyric acid by weight. The composition may comprise a combined total of 80% to 99.9% carbohydrate plus protein in addition to about 0.1% to about 20% beta-hydroxy-beta-methylbutyric acid by weight. The composition may comprise a combined total of 80% to 99.9% protein, carbohydrate, and fat in addition to about 0.1% to about 20% beta-hydroxy-beta-methylbutyric acid by weight.

The composition of the second exemplary embodiment comprises beta-hydroxy-beta-methylbutyric acid for use in increasing dopamine homeostasis in the brain of an obese individual wherein the use results in improvement of at least one of locomotion, mobility and cognition in the individual. The composition may be a nutritional composition. The composition is particularly effective when the individual has consumed a high fat diet.

The composition of the third exemplary embodiment as disclosed herein comprises an amount of HMB that is sufficient and effective to improve a pediatric individual's endurance. The composition may be a nutritional composition. The composition includes an effective amount of HMB to improve endurance of a human subject between the ages of 1 year old and 13 years old, including without limitation, 1-2 years old, 2-3 years old, 3-4 years old, 4-5 years old, 5-6 years old, 6-7 years old, 7-8 years old, 8-9 years old, 9-10 years old, 10-11 years old, 11-12 years old, 12-13 years old, etc.

In certain exemplary embodiments the composition is a nutritional composition comprising from about 10% to about 15% protein; from about 30% to about 50% carbohydrate; and from about 30% to about 50% fat. In certain exemplary embodiments the composition is a nutritional composition comprising from about 10% to about 15% protein; from about 40% to about 50% carbohydrate; and from about 40% to about 50% fat. The composition comprises beta-hydroxy-beta-methylbutyric acid in an amount effective to improve physical endurance in said subject.

In certain exemplary embodiments the composition comprises about 12% protein; about 44% carbohydrate; and about 44% fat; wherein said composition further comprises beta-hydroxy-beta-methylbutyric acid in an amount effective to improve physical endurance in said subject. The improvement in physical endurance is greater than the improvement in physical endurance seen when the nutritional formula is supplemented with the same concentration of leucine instead of beta-hydroxy-beta-methylbutyric acid.

In certain embodiments the composition comprises from about 0.1% to about 20% by weight beta-hydroxy-beta-methylbutyric acid %, including from about 0.1% to about 8%, and from about 0.1% to about 2%, including from about 0.1% to about 5%, including from about 0.3% to about 3%, and also including from about 0.34% to about 1.5%.

In the fourth exemplary embodiment, a pediatric nutritional composition comprises calcium beta-hydroxy-beta-methylbutyric acid, whey protein, casein protein, soy protein, medium chain triglyceride oil, and fructooligosaccharides. The pediatric nutritional composition may optionally comprise other ingredients as described herein.

In certain embodiments of the first, second, third, and fourth exemplary embodiments, the composition comprises from about 12% to about 16% protein; from about 58% to about 62% carbohydrate; and from about 15% to about 20% fat, by weight of the composition.

In certain embodiments of the first, second, third, and fourth exemplary embodiments, the composition comprises from about 0.1% to about 0.5% beta-hydroxy-beta-methylbutyric acid by weight. The compositions of the first, second, third, and fourth exemplary embodiments as disclosed herein may comprise HMB, which means that the compositions are either formulated with the addition of HMB, most typically as a calcium monohydrate, or are otherwise prepared so as to contain HMB in the finished product. Any source of HMB is suitable for use herein provided that the finished product contains HMB, although such a source is preferably calcium HMB and is most typically added as such during product formulation.

In certain embodiments of the first, second, third, and fourth exemplary embodiments, the beta-hydroxy-beta-methylbutyric acid is present as calcium beta-hydroxy-beta-methylbutyric acid. Calcium HMB monohydrate is commercially available from Technical Sourcing International (TSI) of Salt Lake City, Utah and from Lonza Group Ltd. (Basel, Switzerland). Although calcium HMB monohydrate is one preferred source of HMB for use in the first, second, third, and fourth exemplary embodiments, other suitable sources may be used, including HMB as the free acid, a salt, an anhydrous salt, an ester, a lactone, or other product form that otherwise provides a bioavailable form of HMB for the nutritional product. Non-limiting examples of suitable salts of HMB for use in the first, second, third, and fourth exemplary embodiments include HMB salts, hydrated or anhydrous, of sodium, potassium, magnesium, chromium, calcium, or other non-toxic salt form.

In certain embodiments of the first, second, third, and fourth exemplary embodiments, it has been found that high concentrations of leucine are not required to achieve improved physical endurance, locomotion, mobility or cognition. In certain exemplary embodiments of the first, second, third, and fourth embodiments, the composition is substantially free of free leucine.

In certain embodiments of the first, second, third, and fourth exemplary embodiments the composition is a liquid, in which case the concentration of HMB in the liquid may range up to about 10%, including from about 0.1% to about 8%, including from about 0.1% to about 2%, including from about 0.1% to about 5%, including from about 0.3% to about 3%, and also including from about 0.34% to about 1.5%, by weight of the liquid. In certain embodiments of the first, second, third, and fourth exemplary embodiments, the HMB may be present in the liquid formulation in an amount of from about 0.1% to about 0.5% by weight of the liquid. In certain embodiments of the first, second, third, and fourth embodiments, the composition is a nutritional liquid and comprises from about 1% to about 10% protein; from about 10% to about 20% carbohydrate; and from about 1% to about 10% fat, by weight of the composition. In certain embodiments of the first, second, third, and fourth embodiments, the composition is a nutritional liquid and comprises from about 5% to about 8% protein; from about 15% to about 18% carbohydrate; and from about 5% to about 9% fat, by weight of the composition.

In certain embodiments of the first, second, third, and fourth exemplary embodiments the composition is a solid, in which case the concentration of HMB in the solid may range up to about 15%, including from about 0.1% to about 10%, including from about 0.1% to about 2%, including from about 0.2% to about 5%, including from about 0.3% to about 3%, and also including from about 0.34% to about 1.5%, by weight of the powder. In certain embodiments of the first, second, third, and fourth exemplary embodiments, the HMB is present in the powder formulation in an amount of from about 0.1% to about 0.5% by weight of the powder. In certain embodiments of the first, second, third, and fourth embodiments, the composition is a nutritional solid or a nutritional powder and comprises from about 10% to about 20% protein; from about 50% to about 70% carbohydrate; and from about 10% to about 25% fat, by weight of the composition.

### Product Form

The compositions of the first, second, third and fourth exemplary embodiments include HMB. The compositions may be formulated and administered in any known or otherwise suitable oral product form. Any solid, semi-solid, liquid, semi-liquid, or powder form, including combinations or variations thereof, are suitable for use herein, provided that such form allows for safe and effective oral delivery to the individual of the ingredients as defined herein.

The compositions of the first, second, third and fourth exemplary embodiments may be formulated to include only the ingredients described herein, or may be modified with optional ingredients to form a number of different product forms. The exemplary compositions disclosed herein are preferably formulated as dietary product forms, which are defined herein as those embodiments comprising the ingredients disclosed herein in a product form that contains at least one of protein, carbohydrate, fat, and preferably also contains vitamins, minerals, and combinations thereof.

The compositions of the first, second, third, and fourth exemplary embodiments may therefore include a variety of different product forms, including most any conventional or otherwise known food product form, some non-limiting examples of which include confectionary products, cereals, food condiments (e.g., spreads, powders, sauces, jams, jelly, coffee creamer or sweetener), pasta, baking or cooking materials (e.g., flour, fats or oils, butter or margarine, breading or baking mixes), salted or seasoned snacks, extruded, baked, or fried goods, beverages (e.g., coffee, juice, carbonated beverages, non-carbonated beverages, tea, ice-cream based drinks), snack or meal replacement bars (e.g., Slimfast™ bars, Ensure™ bars, Zone Perfect™ bars, Glucerna™ bars), smoothies, breakfast cereals, cheeses, gummy products, salted or unsalted crisp snacks (e.g., chips, crackers, pretzels), dips, baked goods (e.g., cookies, cakes, pies, pastries, bread, bagels, croutons, dressings, dry mixes (e.g., mixes for muffins, cookies, waffles, pancakes, beverages), frozen desserts (e.g., ice cream, popsicles, fudge bars, crushed ice, frozen yogurt), processed meats (e.g., corndogs, hamburgers, hotdogs, sausage, pepperoni), pizza, pudding, flavored or unflavored gelatin, refrigerated dough (e.g., cookies, bread, brownies), milk or soy-based smoothies, yogurt or yogurt-based drinks, frozen yogurt, soy milk, soups, vegetable-based burgers, and popcorn-based snacks.

The compositions of the first, second, third, and fourth exemplary embodiments, when formulated as a dietary product form, may potentially provide either a sole source or a supplemental source of nutrition to an individual. In this context, a sole source of nutrition is one that can be administered once or multiple times each day to potentially provide an individual with all or substantially all their protein, carbohydrate, fat, mineral, and vitamin needs per day or during the intended period of administration. A supplemental source of nutrition is defined herein as a dietary source that does not provide an individual with a potentially sole source of nutrition.

The compositions of the first, second, third, and fourth exemplary embodiments may also be formulated in product forms such as capsules, tablets, pills, caplets, gels, liquids (e.g., suspensions, solutions, emulsions, clear solutions), powders or other particulates, and so forth. These product forms generally contain only the ingredients as described herein, optionally in combination with other actives, processing aids or other dosage form excipients.

The compositions of the first, second, third, and fourth exemplary embodiments may be formulated as milk-based liquids, soy-based liquids, low-pH liquids, clear liquids, reconstitutable powders, nutritional bites (e.g., plurality of smaller dietary product dosage forms in a single package), or nutritional bars (snack or meal replacement).

### Macronutrients

The compositions of the first, second, third, and fourth exemplary embodiments may comprise one or more optional macronutrients in addition to the HMB described herein. The optional macronutrients may include proteins, carbohydrates, fats, and combinations thereof. The compositions of the first, second, third, and fourth exemplary embodiments may be formulated as dietary products containing all three macronutrients.

Macronutrients suitable for use in the first, second, third, and fourth exemplary embodiments may include any protein, carbohydrate, fat, or source thereof that is known for or otherwise suitable for use in an oral composition, provided that the optional macronutrient is safe and effective for oral administration and is otherwise compatible with the other ingredients in the composition.

The concentration or amount of optional protein, carbohydrate, or fat in the composition of the first, second, third, and fourth exemplary embodiments may vary considerably depending upon the particular product form (e.g., bars or other solid dosage forms; milk or soy based liquids; clear beverages; reconstitutable powders, gels, puddings, etc.) and the various other formulations and targeted dietary needs. These optional macronutrients are most typically formulated within any of the exemplary ranges described in Tables 1 and 2 below.

TABLE 1

| Nutrient (% total | | | |
|---|---|---|---|
| calories) | Example A | Example B | Example C |
| Carbohydrate | 0-100 | 10-70 | 40-50 |
| Fat | 0-100 | 20-65 | 35-55 |
| Protein | 0-100 | 5-40 | 15-25 |

Each numerical value in Table 1 is preceded by the term “about”.

TABLE 2

| Nutrient (wt % | | | |
|---|---|---|---|
| composition) | Example D | Example E | Example F |
| Carbohydrate | 0-98 | 1-50 | 10-30 |
| Fat | 0-98 | 1-30 | 1-15 |
| Protein | 0-98 | 1-30 | 1-10 |

Each numerical value in Table 2 is preceded by the term “about”.

### Protein

Proteins suitable for use in the compositions of the first, second, third, and fourth exemplary embodiments may include hydrolyzed, partially hydrolyzed or non-hydrolyzed proteins or protein sources, and can be derived from any known or otherwise suitable source such as milk (e.g., casein, whey), animal (e.g., meat, fish, egg albumen), cereal (e.g., rice, corn), vegetable (e.g., soy, pea, potato), and combinations thereof. The proteins for use herein may also include, or be entirely or partially replaced by, free amino acids known for use in the compositions of the first, second, third, and fourth exemplary embodiments, non-limiting examples of which include L-tryptophan, L-glutamine, L-tyrosine, L-methionine, L-cysteine, taurine, L-arginine, L-carnitine, and combinations thereof.

The compositions of the first, second, third, and fourth exemplary embodiments may optionally comprise a soy protein component, sources of which include, but are not limited to, soy flakes, soy protein isolates, soy protein concentrate, hydrolyzed soy protein, soy flour, soy protein fiber, or any other protein or protein source derived from soy. Commercial sources of soy protein are well known in the nutrition art, some non-limiting examples of which include soy protein isolates distributed by The Solae Company (St. Louis, Mo.) under the trade designation “Soy Protein Isolate EXP-H0118,” “EXP-E-0101, and “Supro Plus 675.” The optional soy protein component may represent from zero to about 100%, or from about 10% to 100%, and including from about 15% to about 100%, including from about 75% to about 95%, and also including from about 80% to about 90% of the total protein calories in the composition.

The compositions of the first, second, third, and fourth exemplary embodiments may therefore, and desirably, further comprise a protein in addition to the HMB, wherein the solid forms of the exemplary compositions generally comprise protein in addition to the HMB in quantities ranging up to about 30%, including from about 5% to about 25%, including from about 10% to about 20%, and also including from about 12% to about 16%, by weight of the solid composition.

For liquid forms of the exemplary compositions, the compositions generally comprise protein in quantities ranging up to about 30%, including from about 1% to about 20%, including from about 1% to about 10%, and also including from about 5% to about 8%, by weight of the liquid composition.

### Carbohydrate

Carbohydrates suitable for use in the compositions of the first, second, third, and fourth exemplary embodiments may be simple, complex, or variations and combinations thereof, all of which are optional, in addition to the HMB as described herein. Non-limiting examples of suitable carbohydrates include hydrolyzed or modified starch or cornstarch, maltodextrin, isomaltulose, sucromalt, glucose polymers, sucrose, corn syrup, corn syrup solids, rice-derived carbohydrate, glucose, fructose, lactose, high fructose corn syrup, honey, sugar alcohols (e.g., maltitol, erythritol, sorbitol), and combinations thereof.

Carbohydrates suitable for use herein may include soluble dietary fiber, non-limiting examples of which include gum arabic, fructooligosaccharide (FOS), sodium carboxymethyl cellulose, guar gum, citrus pectin, low and high methoxy pectin, oat and barley glucans, carrageenan, psyllium and combinations thereof. Insoluble dietary fiber may also be suitable as a carbohydrate source herein, non-limiting examples of which include oat hull fiber, pea hull fiber, soy hull fiber, soy cotyledon fiber, sugar beet fiber, cellulose, corn bran, and combinations thereof.

The compositions of the first, second, third, and fourth exemplary embodiments may therefore, and desirably, further comprise a carbohydrate in addition to the HMB, wherein for solid forms of the exemplary compositions disclosed herein, the compositions generally comprise carbohydrates in addition to the HMB in quantities ranging up to about 75%, including from about 20% to about 70%, including from about 50% to about 70%, including from about 55% to about 65%, and also including from about 58% to about 62%, by weight of the solid composition.

For liquid embodiments of the compositions of the first, second, third, and fourth exemplary embodiments, the liquid embodiments generally comprise carbohydrate in addition to the HMB in quantities ranging up to about 30%, including from about 5% to about 25%, including from about 10% to about 20%, and also including from about 15% to about 18%, by weight of the liquid composition.

### Fat

Fats suitable for use in the compositions of the first, second, third, and fourth exemplary embodiments include coconut oil, fractionated coconut oil, soy oil, corn oil, olive oil, safflower oil, high oleic safflower oil, high GLA-safflower oil, MCT oil (medium chain triglycerides), sunflower oil, high oleic sunflower oil, palm and palm kernel oils, palm olein, canola oil, marine oils, flaxseed oil, borage oil, cottonseed oils, evening primrose oil, blackcurrant seed oil, transgenic oil sources, fungal oils, marine oils (e.g., tuna, sardine), and so forth.

The compositions of the first, second, third, and fourth exemplary embodiments may optionally comprise a flaxseed component, non-limiting examples of which include ground flaxseed and flaxseed oil. In one exemplary embodiment, the flaxseed component is ground flaxseed. Non-limiting examples of flaxseed include red flaxseed, golden flaxseed, and combinations thereof. In one exemplary embodiment, the flaxseed component is golden flaxseed. Commercial sources of flaxseed are well known in the nutrition and formulation arts, some non-limiting examples of which include flaxseed and flax products available from the Flax Council of Canada, the Flax Consortium of Canada, and Heintzman Farms (North Dakota) (Dakota Flax Gold brand).

The compositions of the first, second, third, and fourth exemplary embodiments may therefore, and desirably, further comprise a fat in addition to the HMB, wherein for solid forms of the exemplary compositions, the compositions generally comprise fat in addition to the HMB in quantities ranging up to about 35%, including from about 5% to about 30%, including from about 10% to about 25%, and also including from about 15% to about 20%, by weight of the solid composition.

For liquid forms of the exemplary compositions, the compositions generally comprise fat in addition to the HMB in quantities ranging up to about 30%, including from about 1% to about 20%, including from about 1% to about 10%, and also including from about 5% to about 9%, by weight of the liquid composition.

### Other Optional Ingredients

The compositions of the first, second, third, and fourth exemplary embodiments may further comprise other optional components that may modify the physical, chemical, aesthetic or processing characteristics of the compositions or serve as pharmaceutical or additional components when used in the targeted population. Many such optional ingredients are known or otherwise suitable for use in compositions or pharmaceutical dosage forms and may also be used in the compositions of the first, second, third, and fourth exemplary embodiments as disclosed or otherwise suggested herein, provided that such optional ingredients are safe and effective for oral administration and are compatible with the other selected ingredients in the composition.

Non-limiting examples of such other optional ingredients include preservatives, anti-oxidants, buffers, additional pharmaceutical actives, sweeteners including artificial sweeteners (e.g., saccharine, aspartame, acesulfame K, sucralose), colorants, flavors, branch chain amino acids, essential amino acids, free amino acids, flavor enhancers, thickening agents and stabilizers, emulsifying agents, lubricants, and so forth.

The compositions of the first, second, third, and fourth exemplary embodiments may comprise one or more minerals, non-limiting examples of which include phosphorus, sodium, chloride, magnesium, manganese, iron, copper, zinc, iodine calcium, potassium, chromium (e.g., chromium picolinate), molybdenum, selenium, and combinations thereof.

The compositions of the first, second, third, and fourth exemplary embodiments may also comprise one or more vitamins, non-limiting examples of which include carotenoids (e.g., beta-carotene, zeaxanthin, lutein, lycopene), biotin, choline, inositol, folic acid, pantothenic acid, choline, vitamin A, thiamine (vitamin B1), riboflavin (vitamin B2), niacin (vitamin B3), pyridoxine (vitamin B6), cyanocobalamine (vitamin B12), ascorbic acid (vitamin C), vitamin D, vitamin E, vitamin K, and various salts, esters or other derivatives thereof, and combinations thereof.

In some exemplary embodiments, the compositions disclosed herein comprise both vitamins and minerals.

### Methods of Using the HMB-Containing Compositions

The compositions including HMB of the first, second, third, and fourth exemplary embodiments can be used in various methods as set forth herein for pediatric individuals. These methods include the oral administration of the beta-hydroxy-beta-methylbutyric acid-containing compositions to an individual to improve endurance, locomotion, mobility, and/or cognition in the individual, including a pediatric individual.

In some exemplary embodiments, the individual consumes at least one serving daily of the composition of the first, second, third, or fourth exemplary embodiments. In some exemplary embodiments, the individual consumes two, three, or even more servings per day. Each serving is desirably administered as a single, undivided dose, although the serving may also be divided into two or more partial or divided servings to be taken at two or more times during the day. The compositions of the first, second, third, and fourth exemplary embodiments for use in the methods include continuous day after day administration, as well as periodic or limited administration, although continuous day after day administration is generally desirable. In some exemplary embodiments, the compositions of the first, second, third, and fourth exemplary embodiments for use in the methods are applied on a daily basis, wherein the daily administration is maintained continuously for at least 3 days, including at least 5 days, including at least 1 month, including at least 6 weeks, including at least 8 weeks, including at least 2 months, and including at least 6 months. In some exemplary embodiments, the compositions are administered for 18-24 months as a long term, continuous, daily, dietary supplement.

The compositions of the first, second, third, and fourth embodiments for use in the methods are also intended to include the use of such methods in individuals unaffected by or not otherwise afflicted by decreased endurance, for the purpose of preventing, minimizing, or delaying the development of such conditions involving endurance over time. For such prevention purposes, the methods disclosed herein preferably include continuous, daily administration of the exemplary compositions described herein.

### Method of Manufacture

The compositions of the first, second, third, and fourth exemplary embodiments may be prepared by any known or otherwise effective manufacturing technique for preparing the selected product form. Many such techniques are known for any given product form such as nutritional liquids, nutritional powders, or nutritional bars and can easily be applied by one of ordinary skill in the nutrition and formulation arts to the nutritional products described or otherwise suggested herein.

The compositions of the second and third exemplary embodiment as disclosed herein may likewise be prepared by any known or otherwise effective manufacturing technique for preparing the selected product form. The compositions of the second and third exemplary embodiments as disclosed herein may be in the form of a capsule, tablet, caplet, pill, liquid, suspension, emulsion, gel, and combinations thereof. Many such techniques are well known, for example in the pharmaceutical industry, and can be applied by one of ordinary skill in the nutrition and formulation arts to produce forms such as capsules, tablets, caplets, pills, liquids (e.g., suspensions, emulsions, gels, solutions), and so forth, and can easily be applied by one of ordinary skill in those arts to the non-dietary products described herein.

The compositions of the first, second, third, and fourth exemplary embodiments may be liquid, milk or soy-based nutritional liquids. The compositions of the first, second, third, and fourth exemplary embodiments may be prepared by first forming an oil and fiber blend containing all formulation oils, any emulsifier, fiber and fat-soluble vitamins. Additional slurries (such as a carbohydrate and two protein slurries) are prepared separately by mixing the HMB, carbohydrate and minerals together and the protein in water. The slurries are then mixed together with the oil blend. The resulting mixture is homogenized, heat processed, standardized with any water-soluble vitamins and flavors after which and the liquid is terminally sterilized and aseptically filled or dried (e.g., by spray drying) to produce a powder.

The compositions of the first, second, third, and fourth exemplary embodiments may be in other product forms such as nutritional bars. These compositions may be manufactured, for example, using cold extrusion technology as is known and commonly described in the bar manufacturing art. To prepare such compositions, typically all of the powdered components are dry blended together, which typically includes any proteins, vitamin premixes, certain carbohydrates, and so forth. The fat-soluble components are then blended together and mixed with any powdered premixes. Finally, any liquid components are then mixed into the composition, forming a plastic like composition or dough. The resulting plastic mass can then be shaped, without further physical or chemical changes occurring, by cold forming or extrusion, wherein the plastic mass is forced at relatively low pressure through a die, which confers the desired shape. The resultant extrudate is then cut off at an appropriate position to give products of the desired dimensions and weight. If desired, the solid product is then coated, to enhance palatability, and packaged for distribution.

The solid compositions of the first, second, third, and fourth exemplary embodiments may also be manufactured through a baked application or heated extrusion to produce solid product forms such as cereals, cookies, crackers, and similar other product forms. One knowledgeable in the nutrition manufacturing arts would be able to select one of the many known or otherwise available manufacturing processes to produce the desired final product.

The compositions of the first, second, third, and fourth exemplary embodiments may also be manufactured by other known or otherwise suitable techniques not specifically described herein without departing from the spirit and scope of the general inventive concept. The disclosed exemplary embodiments are, therefore, to be considered in all respects as illustrative and not restrictive and any and all changes and equivalents also come within the description of the present disclosure and the general inventive concept upon which it is based. The following non-limiting examples further illustrate the compositions and methods of the present disclosure.

### EXAMPLES

The following Examples provide data and/or illustrate specific embodiments and/or features of the compositions and methods of the first, second, third, and fourth exemplary embodiments disclosed herein. The Examples are given solely for the purpose of illustration and are not to be construed as limitations, as many variations thereof are possible without departing from the spirit and scope of the general inventive concept.

### Example 1

Mice (Strain: C57BL/6J) were weaned at day 21 post birth and divided into 4 groups of 7 animals each. Group 1 animals were put on chow diet and groups 2 to 4 were fed a pediatric nutritional supplement alone or a pediatric nutritional supplement containing either leucine or HMB as given below:

Group 2: Pediatric nutritional supplement alone;

Group 3: Pediatric nutritional supplement with leucine at 10 mg/g; and

Group 4: Pediatric nutritional supplement with HMB at 3.4 mg/g.

All the animals were acclimatized to running on a treadmill for the first 2 weeks. On week 6 animals were run on a treadmill until exhaustion. The average distances run by each group of animals during 5 days of the week (Monday to Friday) were recorded and statistical significance calculated using ANOVA followed by Tukey's Multiple comparison test.

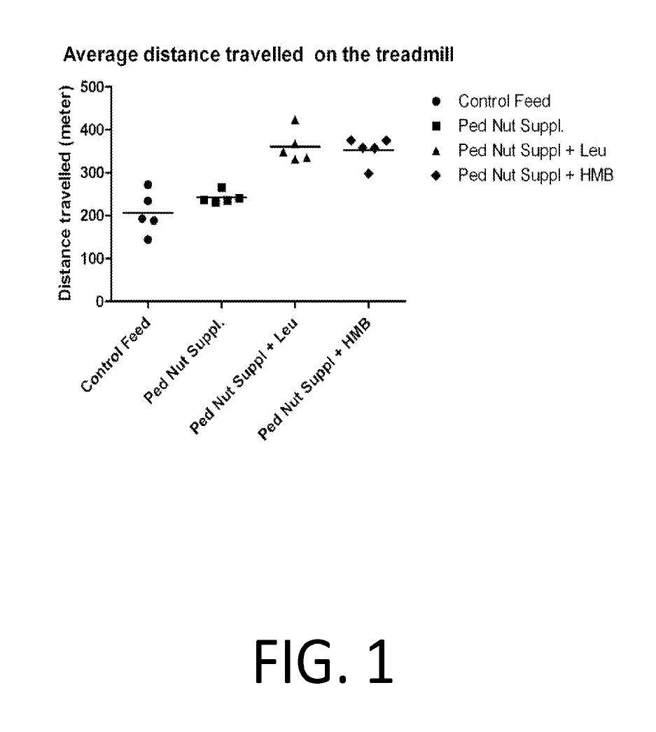

As illustrated in FIG. 1 and Table 3 below, the distance run by the group fed the pediatric nutritional supplement plus leucine was statistically equal to the distance run by the group fed the pediatric supplement plus HMB. Additionally, the group fed the pediatric supplement plus HMB ran farther than the control group or the group fed the pediatric nutritional supplement alone.

TABLE 3

| Tukey's Multiple | Mean | | Significant? | |
|---|---|---|---|---|
| Comparison Test | Diff. | q | P < 0.05? | Summary |
| Control Feed vs Ped | −35.72 | 2.268 | No | ns |
| Nut Suppl. | | | | |
| Control Feed vs Ped | −154.7 | 9.823 | Yes | *** |
| Nut Suppl + Leu | | | | |
| Control Feed vs Ped | −146.4 | 9.294 | Yes | *** |
| Nut Suppl + HMB | | | | |
| Ped Nut Suppl. vs Ped | −119 | 7.555 | Yes | *** |
| Nut Suppl + Leu | | | | |
| Ped Nut Suppl. vs Ped | −110.7 | 7.026 | Yes | *** |
| Nut Suppl + HMB | | | | |
| Ped Nut Suppl +Leu vs | 8.34 | 0.5295 | No | ns |
| Ped Nut Suppl + HMB | | | | |

### Example 2

The mice arrived at the facility at 21 days post birth and were divided into 6 groups after a seven day quarantine. There were 5 animals each. Groups 1 through 4 were placed on the following diets:

Group 1: High Fat Diet alone;

Group 2: High Fat Diet plus 500 mg of leucine/kg of drinking water;

Group 3: High Fat Diet plus 1000 mg of leucine/kg of drinking water;

Group 4: High Fat Diet plus 170 mg of Ca-HMB/kg of drinking water;

Group 5: High Fat Diet plus 340 mg of Ca-HMB/kg of drinking water; and

Group 6: AIN-93G diet.

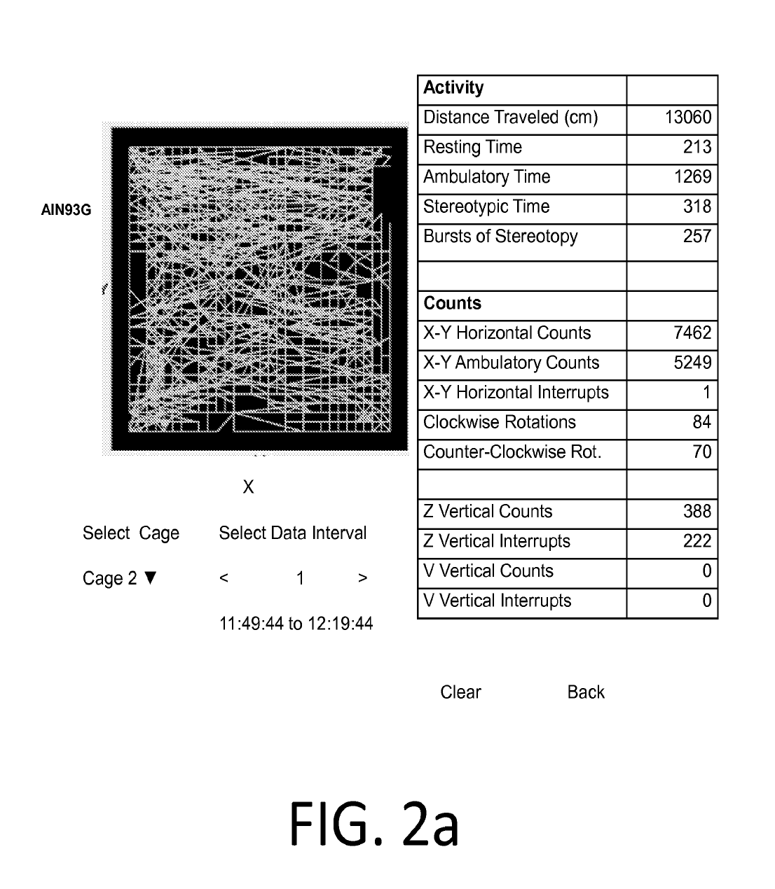

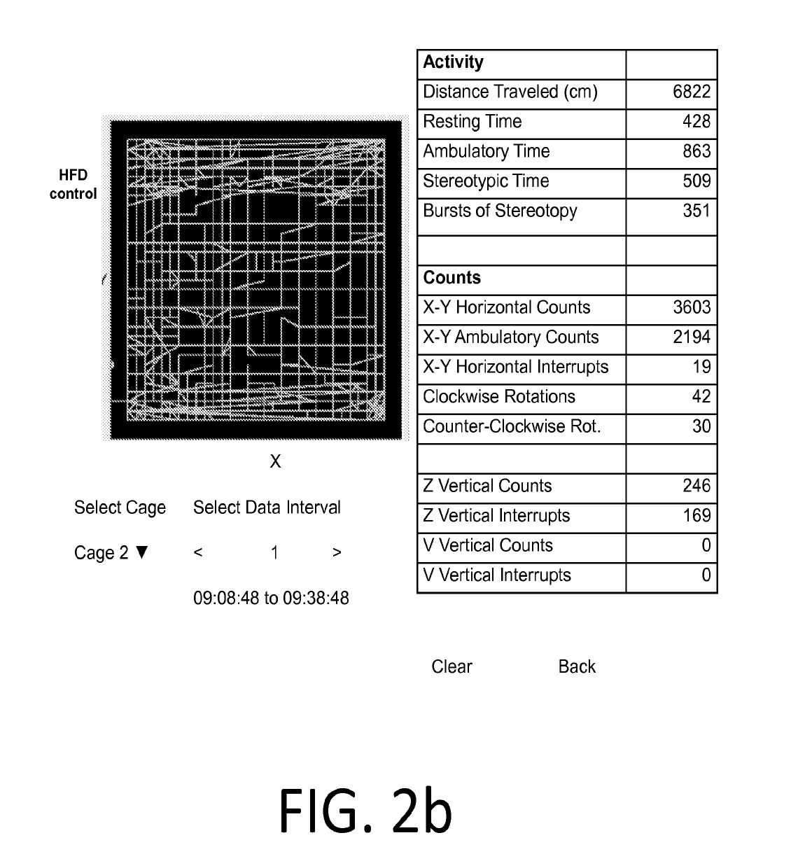

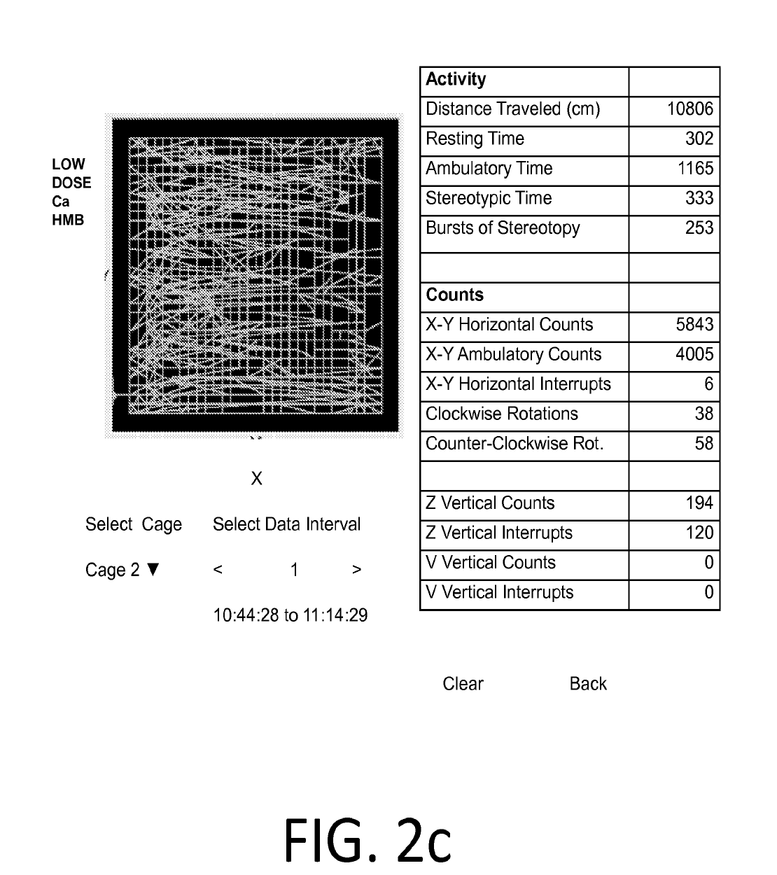

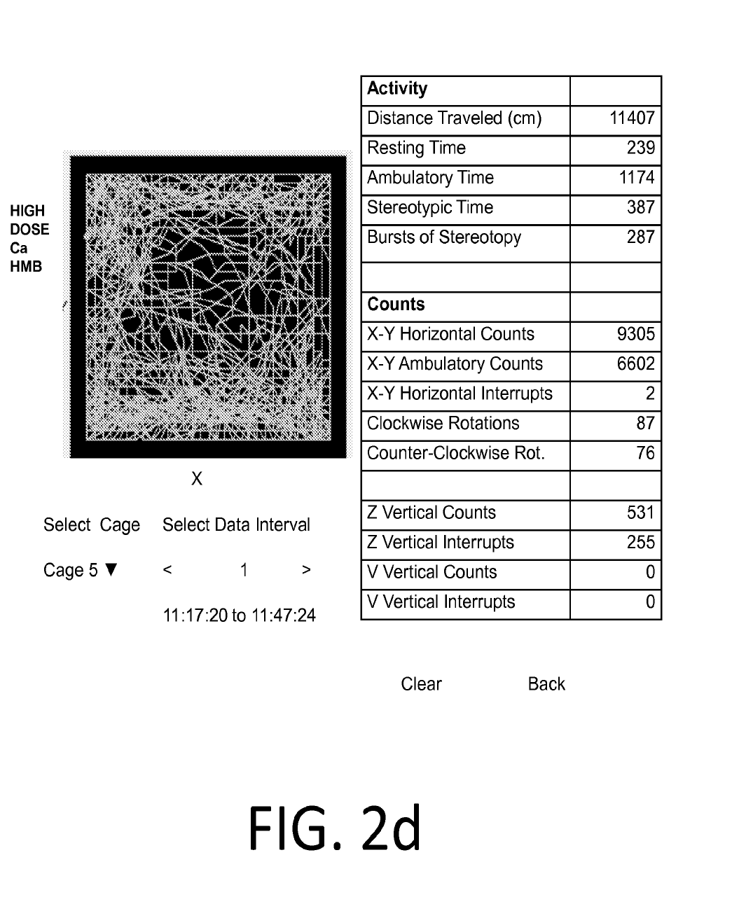

One week before the initiation of the study, the animals were acclimatized in the activity monitoring chamber, daily for half an hour. On the day of experiment, each set of animals were placed in the activity monitoring chamber (chamber 1 to 5) for half an hour. Motor activity of the mice in each group was monitored using the Actometry Apparatus: “Opto-Varimex 4” Auto-track system, Model No: 0271-004M. The motor activity of each mouse was monitored once in a week between 8:30 AM and 12:00 PM for 30 minutes. Six sets of “half an hour observations” were made for each of the 30 animals. The software captured motor activity parameters while the animals were in the activity monitoring chamber. The results were compiled for each group and expressed as Mean±SEM for each parameter as shown in FIGS. 2a-2d.

As illustrated in FIGS. 2a-2d, mice fed a high fat diet plus HMB demonstrated enhanced exploratory activity compared to mice fed the high fat diet alone.

### Examples 3-7

### Exemplary Compositions

| | Amount per 1000 kg | | | | |
|---|---|---|---|---|---|
| Ingredients | Ex. 3 | Ex. 4 | Ex. 5 | Ex. 6 | Ex. 7 |
| Water | Q.S. | Q.S. | Q.S. | Q.S. | Q.S. |
| Calcium HMB | 3.4 kg | 3.2 kg | 3.0 kg | 3.7 kg | 4.25 kg |
| Corn Maltodextrin | 63.4 kg | 63.4 kg | 63.4 kg | 63.4 kg | 63.4 kg |
| Sugar | 60.2 kg | 60.2 kg | 60.2 kg | 60.2 kg | 60.2 kg |
| Milk Protein | 24.6 kg | 24.6 kg | 24.6 kg | 24.6 kg | 24.6 kg |
| Concentrate | | | | | |
| High Oleic | 15.6 kg | 15.6 kg | 15.6 kg | 15.6 kg | 15.6 kg |
| Safflower Oil | | | | | |
| Soy Oil | 15.3 kg | 15.3 kg | 15.3 kg | 15.3 kg | 15.3 kg |
| Whey Protein | 5.52 kg | 5.52 kg | 5.52 kg | 5.52 kg | 5.52 kg |
| Concentrate | | | | | |
| Medium Chain | 5.46 kg | 5.46 kg | 5.46 kg | 5.46 kg | 5.46 kg |
| Triglycerides | | | | | |
| Soy Protein Isolate | 4.92 kg | 4.92 kg | 4.92 kg | 4.92 kg | 4.92 kg |
| Fructooligosaccharides | 4.45 kg | 4.45 kg | 4.45 kg | 4.45 kg | 4.45 kg |
| Potassium Hydroxide | 2.41 kg | 2.41 kg | 2.41 kg | 2.41 kg | 2.41 kg |
| Potassium Citrate | 1.42 kg | 1.42 kg | 1.42 kg | 1.42 kg | 1.42 kg |
| Magnesium Phosphate | 1.19 kg | 1.19 kg | 1.19 kg | 1.19 kg | 1.19 kg |
| Flavor | 1.10 kg | 1.10 kg | 1.10 kg | 1.10 kg | 1.10 kg |
| Microcrystalline | 3.00 kg | 3.00 kg | 3.00 kg | 3.00 kg | 3.00 kg |
| Cellulose | | | | | |
| Calcium Phosphate, | 860 g | 860 g | 860 g | 860 g | 860 g |
| Tribasic | | | | | |
| Potassium Chloride | 859 g | 859 g | 859 g | 859 g | 859 g |
| Sodium Chloride | 689 g | 689 g | 689 g | 689 g | 689 g |
| WSV/TM/UTW Premix | 563 g | 563 g | 563 g | 563 g | 563 g |
| Soy Lecithin | 375 g | 375 g | 375 g | 375 g | 375 g |
| Monoglycerides | 375 g | 375 g | 375 g | 375 g | 375 g |
| Choline Chloride | 337 g | 337 g | 337 g | 337 g | 337 g |
| Potassium Phosphate, | 313 g | 313 g | 313 g | 313 g | 313 g |
| Monobasic | | | | | |
| Ascorbic Acid | 227 g | 227 g | 227 g | 227 g | 227 g |
| Calcium Carbonate | 214 g | 214 g | 214 g | 214 g | 214 g |
| Carrageenan | 150 g | 150 g | 150 g | 150 g | 150 g |
| DHA Oil | 118 g | 118 g | 118 g | 118g | 118 g |
| Vitamin Premix | 88.8 g | 88.8 g | 88.8 g | 88.8 g | 88.8 g |
| Potassium Phosphate, | 70.7 g | 70.7 g | 70.7 g | 70.7 g | 70.7 g |
| Dibasic | | | | | |
| ARA Oil | 35.1 g | 35.1 g | 35.1 g | 35.1 g | 35.1 g |
| Ferrous Sulfate | 34.5 g | 34.5 g | 34.5 g | 34.5 g | 34.5 g |
| L-Carnitine | 17.6 g | 17.6 g | 17.6 g | 17.6 g | 17.6 g |
| Potassium Iodide | 120 mg | 120 mg | 120 mg | 120 mg | 120 mg |

1. (canceled)

2. A nutritional composition comprising beta-hydroxy-beta-methylbutyric acid for use in increasing dopamine homeostasis in the brain of an obese individual wherein the use results in improving at least one of locomotion, mobility or cognition in the individual.

3. (canceled)

4. The nutritional composition of claim 2, further comprising from 10% to 15% protein; from 30% to 50% carbohydrate; and from 30% to 50% fat.

5. The nutritional composition of claim 2, further comprising from 10% to 15% protein; from 40% to 50% carbohydrate; and from 40% to 50% fat.

6.-8. (canceled)

9. The nutritional composition of claim 2, wherein the nutritional composition comprises from 0.1% to 0.5% beta-hydroxy-beta-methylbutyric acid by weight.

10. The nutritional composition of claim 2, wherein the nutritional composition is a liquid and comprises from 1% to 10% protein; from 10% to 20% carbohydrate; and from 1% to 10% fat, by weight of the nutritional composition.

11. The nutritional composition of claim 2, wherein the nutritional composition is a liquid and comprises from 5% to 8% protein; from 15% to 18% carbohydrate; and from 5% to 9% fat, by weight of the nutritional composition.

12. The nutritional composition of claim 2, wherein the nutritional composition is a solid or a powder and comprises from 10% to 20% protein; from 50% to 70% carbohydrate; and from 10% to 25% fat, by weight of the nutritional composition.

13. The nutritional composition of claim 2, wherein the nutritional composition is a solid or a powder and comprises from 12% to 16% protein; from 58% to 62% carbohydrate; and from 15% to 20% fat, by weight of the nutritional composition.

14. The nutritional composition of claim 2, wherein the beta-hydroxy-beta-methylbutyric acid is present as calcium beta-hydroxy-beta-methylbutyric acid.

15. The nutritional composition of claim 2, wherein the nutritional composition is substantially free of free leucine.
